(19) United States
(12) Patent Application Publication
(10) Pub. No.: US 2009/0091453 A1
(43) Pub. Date: Apr. 9, 2009

(54) STORAGE SYSTEM AND STORED ARTICLE ID MANAGEMENT METHOD

(75) Inventors: Masato Ishida, Kyoto (JP); Kenichi Kobayashi, Kyoto (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: MURATA KIKAI KABUSHIKI KAISHA, Kyoto-shi, Kyoto (JP)

(21) Appl. No.: 12/094,072

(22) PCT Filed: Oct. 20, 2006
(86) PCT No.: PCT/JP2006/320915

§ 371 Date: May 16, 2008

(30) Foreign Application Priority Data
Nov. 18, 2005 (JP) 2005-334071

(51) Int. Cl. G08B 13/14

(52) U.S. Cl. 340/572.1

(57) ABSTRACT

A monitor camera (18) is arranged for monitoring an ID unit (42 of a medical staff. The ID of a staff who is opening a storage cabinet (12) is identified and authenticated by a management device (22). When the authentication is successful, a shelf (28) of the storage cabinet (12) is unlocked. An RFID reader (34) is arranged on the storage cabinet (12) so as to read an RF ID of an article in the shelf (28) when the shelf (28) is opened and closed and identify an article placed or removed according to the difference between the ID lists when the shelf is opened and closed. When a medical staff takes a medical product or medical instrument out of the storage cabinet, it is possible to automatically record who took out which medical product. Moreover, it is possible to prevent taking out of a medical product by an unauthorized person.

### TECHNICAL FIELD

The present invention relates to a storage system for managing medical products, medical instruments, files, or the like.

### BACKGROUND ART

In management of medical products, medical instruments or the like, conventionally, IDs of articles are read, and stored using a portable information terminal, and the staff member who took articles is identified using an ID of the portable information terminal. However, in urgent cases, e.g., in emergent medical treatment, this approach involves difficulties. It is not practical to read IDs of articles one by one using the portable information terminal. As a result, medication errors may occur in the case of emergent medical treatment or the like. Records of medical products used in medical treatments are not clear to patients. Further, records indicating who took the medical products or medical instruments, or which medical products or medical instruments were taken are not clear to the staff member who manages the medical products or medical instruments. Consequently, inventory checks for the medical products or medical instruments, and management of checking the used medical products or medical instruments cannot be performed perfectly.

### PROBLEMS TO BE SOLVED BY THE INVENTION

An object of the present invention is to provide a storage system which makes is possible to manage who retrieved or stored what articles.

Another object of the present invention is to make it possible to reliably detect only articles retrieved and stored by an operator this time.

Still another object of the present invention is to prevent retrieval and storage of articles by an operator without authorization.

Still another object of the present invention is to make it possible to easily collect uncollected articles which were retrieved from a depository.

### MEANS FOR SOLVING THE PROBLEMS

According to the present invention, a storage system having a drawer for storing articles is provided. The storage includes means for reading an ID of an operator who opens or closes the drawer; means for reading IDs of articles in the drawer in response to at least closing of the drawer; and means for determining IDs of articles retrieved from, and stored in the drawer when the drawer is closed based on a difference between a list of IDs of articles read last time, and a list of IDs of articles read this time.

Further, according to the present invention, a method of managing IDs of articles stored in a drawer is provided. The method includes the steps of providing a first ID reader adjacent to the drawer for reading an ID of an operator, a second ID reader for reading IDs of articles in the drawer in a state where the drawer is opened, means for storing a list of the IDs of the articles read by the second ID reader, and means for determining a difference between a stored list of the IDs and the list of the IDs of the articles read by the second ID reader this time; determining the ID of the operator who opens and closes the drawer by the first ID reader; reading the IDs of the articles in the drawer by the second ID reader in response to at least closing of the drawer; and determining the IDs of the articles retrieved from, and stored in the articles when the drawer is closed based on a difference between the list of the IDs of the articles read at the time of closing the drawer last time and the list of the IDs of the articles read at the time of closing the drawer this time.

In this specification, it should be noted that description regarding the storage system is applicable to the stored article ID management method.

The IDs of the articles may be read, e.g., each time the drawer is closed. The difference between the list of the IDs of the articles when the drawer was closed last time and the list of IDs when the drawer was closed this time may be used to create a list of IDs of the retrieved and stored articles.

Alternatively, the IDs may be read when the drawer is closed, and also during closing of the drawer, the ID may be read periodically at predetermined time intervals, and stored. In his case, when the drawer is opened and closed next time, the difference from the list of IDs which was created last time is determined.

However, preferably, the article ID reading means reads the IDs of the articles in the drawer, at the time of opening the drawer, and at the time of closing the drawer.

Preferably, a lock for locking the drawer is provided, and unlocking of the lock is performed based on the read ID of the operator.

Further, preferably, a collection station having means for reading IDs of collected articles is provided, and means for extracting IDs of the uncollected articles by comparing IDs of collected articles and the IDs of the articles retrieved from, and stored in the drawer is provided.

### ADVANTAGES OF THE INVENTION

In the present invention, the operator who took out the article from the drawer is identified by the ID of the operator. At least in response to closing of the drawer, IDs of the articles in the drawer are read. By determining the list of IDs of articles read last time and the list of IDs of articles read this time, it is possible to determine the IDs of articles retrieved and stored this time. Thus, it is possible to automatically check who retrieved or stored what article, and it is possible to omit the time consuming operation of bringing each article to the ID reader for reading the ID of the article. Further, the operator does not have to carry the terminal in one to one association with the operator.

By determining the difference between the list of IDs of the articles at the time of opening the drawer and the list of IDs of the articles at the time of closing the drawer, it is possible to reliably detect only the ID of the article retrieved from and stored in the drawer this time. In particular, at the time of opening the drawer and at the time of closing the drawer, by scanning an area inside the drawer in response to the opening or closing operation of the drawer to read the IDs of the articles, it is possible to read the IDs of the article further reliably.

By unlocking the lock of the drawer based on the read ID of the operator, it is possible to prevent articles from being retrieved from, or stored in the drawer by a person without authorization.

Further, by comparing the IDs of the articles collected to the collection box and the IDs of the articles taken out of the drawer, it is possible to identify the uncollected articles which were retrieved from the drawer, but have not been collected. Thus, it is possible to easily check the state after the articles were taken out of the drawer.

### BRIEF DESCRIPTION OF THE DRAWINGS

FIG. 1 is a diagram showing a layout of a storage system according to an embodiment.

FIG. 2 is a view schematically showing retrieval and storage of an article with an RFID from a depository according to the embodiment.

FIG. 3 is a flow chart showing an algorithm for management of articles in a medical treatment room according to the embodiment.

FIG. 4 is a flow chart showing an algorithm for management of articles in a depository according to the embodiment.

FIG. 5 is a front view showing a wagon used in the embodiment.

### DESCRIPTION OF THE NUMERALS

2: storage system
4: medical treatment room
6: storage room
8: bed
10: medical treatment area
12: depository
14: medical treatment terminal
16: collection box
18: surveillance camera
20: speaker
22: management device
24: inventory file
26: log file
27: uncollected article file
28: drawer
30, 31: article
32: RFID
34, 36: RFID reader
38: depository unit
40: cap
42: ID unit
50: wagon
52: drawer
54: RFID reader

### BEST MODE FOR CARRYING OUT THE INVENTION

Hereinafter, an embodiment in the most preferred form for carrying out the present invention will be described.

### Embodiment

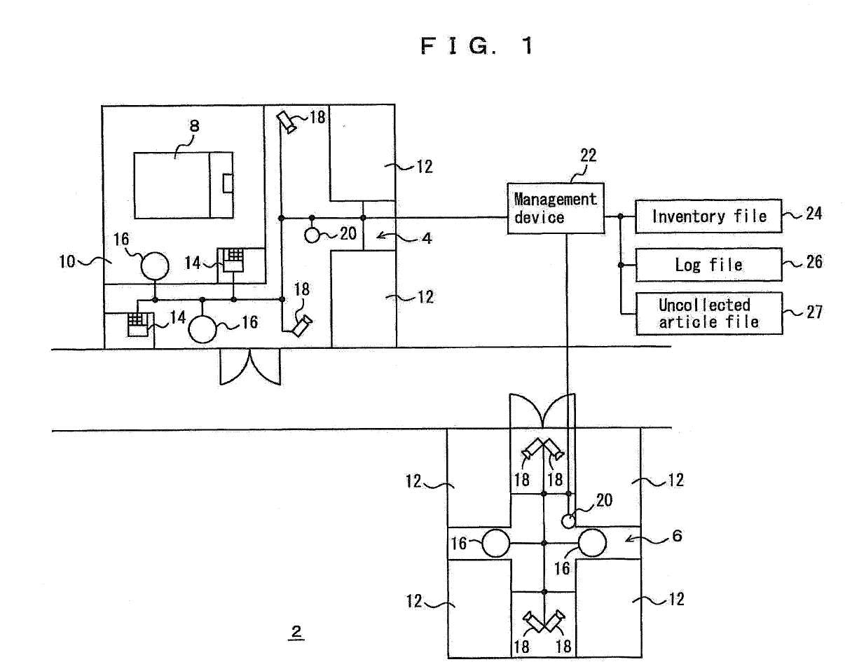

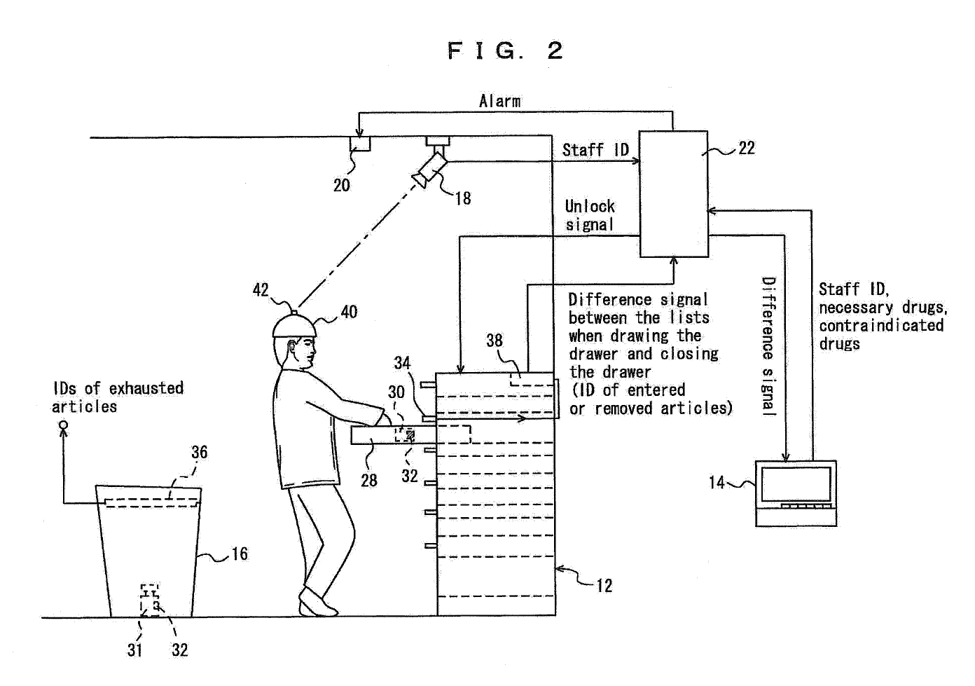

FIGS. 1 to 5 show a storage system 2 according to the embodiment. In the drawings, a reference numeral 4 denotes a medical treatment room, a reference numeral 6 denotes a storage room, a reference numeral 8 denotes a bed of a patient, and a reference numeral 10 denotes a medical treatment area. For example, the medical treatment area 10 is divided by a partition such as an air curtain. Reference numerals 12 denote depositories. The depositories 12 are managed using data of numbers assigned to the respective depositories 12. Each of the depositories 12 has drawers 28, and a drawer number is assigned to each of the drawers 28 in the depositories 12.

Reference numerals 14 denote medical treatment room terminals. Although a plurality of terminals 14 are provided inside and outside of the medical treatment area 10 in the illustrated embodiment, only one terminal 14 may be provided. Any of the medical treatment room terminals 14 can also be used as a management device in the medical treatment room 4. Reference numerals 16 denote collection boxes for collecting used medical products, medical instruments or the like. The shape of the collection box 16 is not limited to the box shape. For example, the collection box 16 may have a depository shape. Reference numerals 18 denote surveillance cameras for reading an ID unit 42 of the medical staff member who retrieves articles from, and stores articles in the depository. Using the surveillance camera 18, the ID of the medical treatment member is read, and the position and the orientation of the staff member, i.e., whether the staff member is oriented toward the depository 12 or not are detected. A reference numeral 20 denotes a speaker. If a wrong medical product or medical instrument is retrieved, the speaker 20 notifies the error, and if a contraindicated drug is retrieved, the speaker 20 issues an alarm.

A reference numeral 22 denotes a management device for managing the entire storage system 2. The management device 22 has an inventory file 24 indicating articles stored in the depositories 12, a log file 26 for records of retrieval and storage of articles from, and into the depositories 12, and an uncollected article file 27 indicating articles which were retrieved from the depositories 12, but have not been collected to the collection boxes 16. The inventory file 24 stores inventory data of medical products or medical instruments in stock, together with IDs of articles, the times when the articles were received by the storage system 2, depository numbers, and drawer numbers. The log file 26 stores records of retrieval of articles from and storage of articles into the depositories 12, together with IDs of retrieved, and stored articles, depository numbers, drawer numbers, IDs of medical staff members who retrieved or stored articles, and times of retrieval and storage of articles. The uncollected article file 27 stores a list of IDs which were retrieved from the depositories 12, but have not been collected to the collection boxes 16.

The depository 12 has a plurality of drawers 28, and each of the drawers 28 can contain a plurality of articles 30. An ID such as an RFID 32 is attached to each of the articles 30. Instead of the RFID 32, a two-dimensional barcode may be used as the ID. For example, an ID reader is provided for each of the drawers 28 or for each of the depositories 12. At the time of drawing (opening) the drawer 28, the RFIDs 32 of articles 30 in a range where the drawer 28 is drawn are read. Likewise, at the time of closing the drawer 28, the RFIDs 32 of the articles 30 are read. By determining the difference between the ID list of articles at the time of drawing the drawer 28 and the ID list of articles at the time of closing the drawer 28, it is possible to determine the list of articles retrieved from, and stored in the drawer 28 by drawing operation of the drawer 28. For example, the RFID reader 34 has an antenna having a plate shape or a linear shape along the upper side of the drawer 28. When the articles 30 pass through a position under the antenna, the RFIDs 32 are read. In the case where the RFID reader 34 can also be used as an RFID writer, it is possible to write data about retrieval of the articles 30 from, or storage of the articles 30 into the drawer 28, in the RFIDs 32.

An RFID reader 36 having a ring-shaped antenna is provided, e.g., near the opening of the collection box 16. At the time of collecting the article 31, the RFID 32 of the article 31 is read. In the case where the readers 34, 36 are also used as writers, for example, when the article 31 is placed in the drawer 28, the ID of the operator and the number assigned to the drawer 28 may be written using the writer 34, and when the article 31 is collected, the RFID reader 36 may write data indicating that the article 31 has been collected, in the RFID 32.

The medical staff member wears a cap 40, and an ID unit 42 is attached to an upper part of the cap 40 or the like. For example, the ID unit 42 includes a pair of LEDs, a power supply for the LEDs, and a drive circuit. By blinking one of the LEDs, the ID is emitted as a visible light signal. Since the pair of LEDs are provided, when the medical staff member wears the cap 40 in a predetermined orientation, by the orientation of the LED, it is possible to recognize the orientation of the medical staff member. For example, it is possible to recognize whether the medical staff member is oriented to the depository 12, or the medical staff member simply passed the side of the depository 12. Further, since the surveillance camera 18 can detect the position of the ID unit 42, it is possible to reliably detect who of the staff opened or closed the drawer 28.

When the surveillance camera 18 detects the ID unit 42, the surveillance camera 18 sends data indicating to which depository 12 the staff member is orientated, and the ID of the staff member to the management device 22. Based on the data of the ID of the staff member sent from the medical treatment room terminal 14 and data of contraindicated drugs or the like, the management device 22 verifies the staff member, and sends an unlocking signal to the depository 12. Thus, a lock (not shown) of the drawer 28 is unlocked. It should be noted that different articles may be stored depending on the depositories 12, and the types of the accessible depositories 12 may be limited for each ID of the staff member. When the lock is unlocked, or when the ID of the staff member is authenticated, the RFID reader 32 is activated. The RFID reader may be driven by a battery such as a solar battery. Alternatively, the RFID reader may be driven by a commercial power supply.

When the medical staff member opens the drawer 28, and closes the drawer 28, an area inside the drawer 28 is scanned by the RFID reader 34 for reading the IDs 32 of the articles 30 in the drawer 28. The difference between the ID lists is determined by a depository unit 38, and sent to the management device 22. The management device 22 sends IDs of medical products or medical instruments to the medical treatment room terminals 14 for allowing the IDs to be displayed on the medical treatment room terminals 14. At the site where emergent medical treatment is carried out, the drawer 28 may be opened or closed violently, and reading by the RFID reader 34 may not be performed in time. Therefore, preferably, deceleration means (not shown) for opening or closing the drawer is provided on the drawer 28 side to limit the speed of opening or closing the drawer 28. Thus, synchronously with opening or closing of the drawer 28, the RFID reader 34 can scan the IDs of articles 30 in the drawer 28.

The IDs of the exhausted articles (collected articles) ready by the RFID reader 36 at the collection box 16 are sent to the management device 22, and the corresponding IDs are deleted from the uncollected article file 27. Further, data of the corresponding IDs are set as “collected” in the log file.

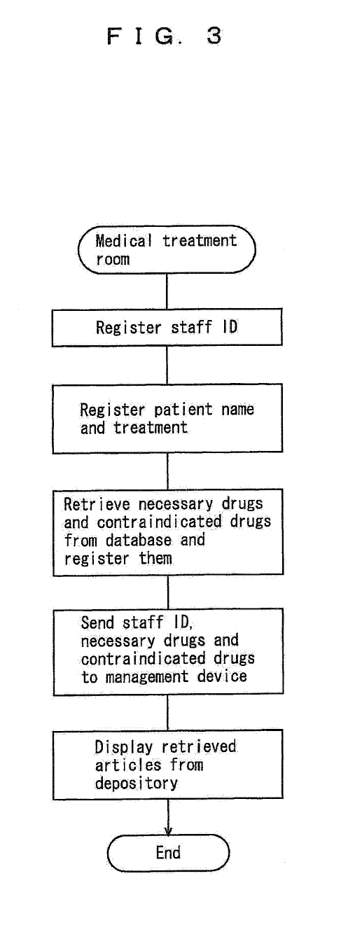

FIG. 3 shows an algorithm of managing an article in the medical treatment room. For example, the IDs of staff are registered, and names of patients and the details of medical treatment are registered. Necessary drugs and contraindicated drugs corresponding to the details of medical treatment are read from a database (not shown), and registered. Likewise, the necessary medical instruments and contraindicated medical instruments are registered. The necessary drugs are drugs required in the medical treatment. The contraindicated drugs are drugs that must not be used in the medical treatment. Instead of registering contraindicated drugs, the drugs with risks may be registered as contraindicated drugs on the management device side, and each time any of the contraindicated drugs becomes required in the medical treatment, registration of the required drug may be deleted from the list of the contraindicated drugs. Data of the IDs of staff members, the necessary drugs, and the contraindicated drugs are transmitted to the management device of the storage system. Conversely, when IDs of the retrieved articles are inputted from the management device, the details of the retrieved articles are displayed on the terminal.

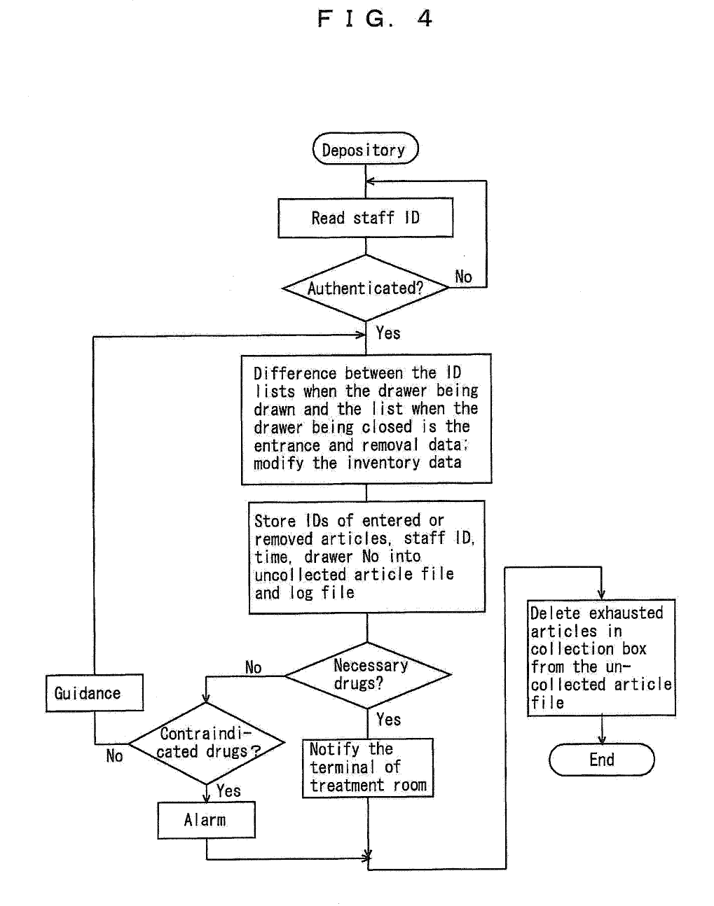

As shown in FIG. 4, on the depository side, the surveillance camera is used to recognize who of the staff is in front of which depository, and check the ID with the ID list of the staff to confirm whether the staff member in question is authorized to retrieve or store the articles. If the staff member is authorized, the depository is unlocked, and the list of articles in the drawer at the time of opening the drawer, and the list of articles in the drawer at the time of closing the drawer are created. The difference between these lists is sent as storage and retrieval data to the management device. Thus, based on the storage and retrieval data, the management device changes the inventory file, and records the IDs of the retrieved articles, the stored articles, and the IDs of staff members, times of retrieval and storage, drawer numbers or the like in the log file and the uncollected article file.

Each of the retrieved medical product and medical instrument are checked whether it is registered as required for the medical treatment or the symptom. If it is not registered as required, and it is not a contraindicated drug, information to this effect is notified, and if it is a contraindicated drug, an alarm is issued. Management of contraindicated drugs is carried out for preventing the use of medical products which must not be used for the medical treatment, and for preventing the contraindicated medical products such as psychoactive drugs from being taken out improperly. By checking whether it is the necessary drug or not, medication errors are prevented. The IDs of the medical products and medical instruments are notified to the terminals in the medical treatment room for making it possible to predict when it is possible to get the necessary drugs, e.g., from the depository outside the medical treatment room.

The IDs of the articles read at the collection box are deleted from the uncollected article file. Data indicting “collected” is set in the IDs of the corresponding articles in the log file. Thus, it is possible to extract articles which were taken out of the drawer, but which have not been collected to the collection box. In the system, it is possible to prevent the unauthorized use or unauthorized taking out of the medical products, and check the conditions of medical products after the taking out from the drawer. Further, it is possible to prevent the use of excessively old medical products after elapse of a long period of time from the taking out from the drawer.

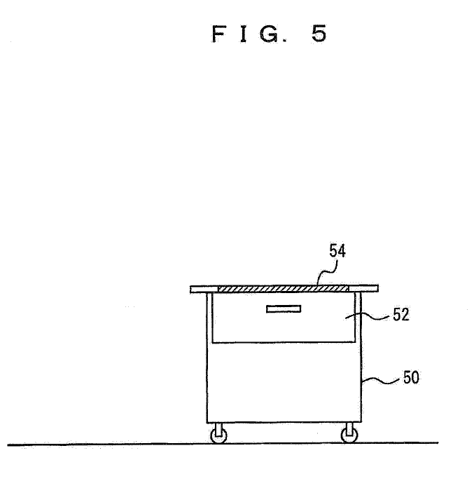

Although the embodiment has been described in connection with the case in which the articles are stored in the depository 12, a wagon 50 as shown in FIG. 5 may be used. A reference numeral 52 denotes a drawer of the wagon 50. A reference numeral 54 denotes an RFID reader for reading RFIDs of articles in the drawer 52 at the time of opening or closing the drawer. Further, in the embodiment, although the visible light ID is used as the ID of the operator, other IDs such as the RFID may be used. In the case where the RFID is used, the ID may be carried by the operator at an arbitrary position.

In the embodiment, the following advantages are obtained.

(1) It is possible to reliably, and automatically check who of the medical staff retrieved or stored which article, from which drawer the medical staff retrieved the article, into which drawer the medical staff stored the article, which article is retrieved from or stored in the drawer, and when the article is retrieved from or stored in the drawer.

(2) Since the difference between IDs of the articles at the time of drawing (opening) the drawer, and IDs of the articles at the time of closing the articles are used, it is possible to reliably detect only the IDs of the articles taken out of or stored in the drawer by opening and closing the drawer. In the case where an RFID reader equipped with an antenna having a plate shape or a wire shape is used, reading of the ID at the time of opening and closing the drawer becomes further easily.

(3) It is possible to prevent the depository from being accessed by a medical staff member without authorization.

(4) Based on the difference between the articles taken out of the drawer and the collected articles, it is possible to identify unused articles or missing articles.

In the embodiment, the IDs of articles are read at the time of opening and closing the drawer. Alternatively, for example, the IDs of the articles in the drawer may be read after the drawer is closed. In this case, the RFID reader is not provided outside the depository 12, but provided inside the depository 12. The IDs of the articles are read after the drawer is closed in the case where it is difficult for the RFID reader to read the IDs while opening or closing movement of the drawer, and no means for limiting the speed of opening and closing the drawer is provided. In this case, the articles retrieved from the drawer, or stored in the drawer are identified by the difference between the ID list created when the drawer was closed last time and the ID list created when the drawer was closed this time. However, in this method, assuming that the drawer is opened and closed in the state where the power supply of the RFID reader is turned off, it is not possible to correctly identify the retrieved or stored articles. Further, if the IDs of the articles in the drawer are read in the state where the drawer is closed, it is not possible to drive the RFID reader by a battery, and wires for driving the RFID reader are required.

Although the embodiment has been described in connection with the case in which management of the medical products and medical instruments is taken as an example, the present invention is also applicable to management of document files. In this case, for example, for storage of document files, a depository having a plurality of drawers are used, and an RFID is provided for each file. At the time of opening and closing the drawer, the IDs of the files are read. Based on the difference of between the IDs read at the time of opening the drawer and the IDs read at the time of closing the drawer, the retrieved and stored files are checked, and IDs of the staff member who opened and closed the drawer is read to identify the staff member.

1. A storage system having at least a drawer for storing articles, the storage system comprising:
means for reading an ID of an operator who opens or closes the drawer;
an RFID reader operated synchronously with each of opening operation and closing operation of the drawer, for reading RFIDs of articles in a range where the drawer is opened or closed, when the articles in the range pass through a position adjacent to the RFID reader by opening or closing of the drawer; and
means for determining RFIDs of articles retrieved from, and stored in the drawer based on a list of RFIDs of articles read at a time of opening the drawer, and a list of RFIDs of articles read at a time of closing the drawer.

2. The storage system according to claim 1, the system being configured to read the RFIDs of the articles by the RFID reader when the articles pass under the RFID reader.

3. The storage system according to claim 2, the RFID reader having an antenna in a plate shape or a linear shape along an upper side of the drawer, the system being configured to read the RFIDs of the articles by the antenna when the articles pass under the antenna.

4. The storage system according to claim 1, further including a lock for locking the drawer, unlocking of the lock being performed based on the read ID of the operator.

5. The storage system according to claim 1, further including a collection station having means for reading RFIDs of collected articles; and means for extracting RFIDs of uncollected articles by comparing the RFIDs of the collected articles and the RFIDs of the articles retrieved from, and stored in the drawer.

6. A method of managing IDs of articles stored in at least a drawer, the method comprising the steps of:
providing a first ID reader adjacent to the drawer for reading IDs of an operator, an RFID reader for reading RFIDs of articles in the drawer in a state where the drawer is opened;
determining the ID of the operator who opens and closes the drawer by the first ID reader;
synchronously with each of opening operation and closing operation of the drawer, reading RFIDs of articles in a range where the drawer is opened or closed by the RFID reader, when the articles in the range where the drawer is opened or closed pass through a position adjacent to the RFID reader by opening or closing of the drawer;
determining RFIDs of the articles retrieved from, and stored in the drawer based on a difference between a list of RFIDs of articles read at the time of opening the drawer, and a list of RFIDs of articles read at the time of closing the drawer.

7. The stored article ID management method according to claim 6, including the step of reading the RFIDs of the articles by the RFID reader when the articles pass under the RFID reader.

8. The stored article ID management method according to claim 7, the RFID reader having an antenna in a plate shape or a linear shape along an upper side of the drawer, reading of the RFID being performed using the antenna when the articles pass under the antenna.
